(19) United States
(12) Patent Application Publication
(10) Pub. No.: US 2009/0234344 A1
(43) Pub. Date: Sep. 17, 2009

(54) Method for the transcutaneous treatment of varicose veins and spider veins using dual laser therapy

(76) Inventor: Timothy Lavender, Pikeville, KY (US)

Correspondence Address: JAMES M. FRANCIS, 300 W VINE ST, STOLL KEENON OGDEN PLLC, LEXINGTON, KY, 40507, US

(21) Appl. No.: 12/046,071

(22) Filed: Mar. 11, 2008

(51) Int. Cl. A61B 18/20

(52) U.S. Cl. 606/10; 128/898; 607/89

(57) ABSTRACT

A method for treating vascular disorders comprising the use of a pulse dye laser followed by the use of an Nd-YAG laser to affect a targeted vessel, said pulse dye laser and said Nd-YAG laser being utilized trans-dermally to said vessel and further comprising the method of using said pulse dye laser to deliver sufficient energy to the blood within said targeted vessel to convert a quantity of Hb to metHb so as to create sufficient heat within said targeted vessel when said metHb is affected by the energy delivered by said Nd-YAG laser.

### BACKGROUND OF THE INVENTION

1. Field of the Invention

This technique relates to the treatment of venous disorders. More specifically this technique relates to the treatment of venous disorders by laser.

2. Problems in the Art

The use of lasers by dermatologists to treat skin disorders is well known in the art. Laser therapy has been used to successfully treat unsightly blemishes, scars, venous disorders, and port wine stains.

The treatment of venous disorders has required the use of a catheter inserted beneath the skin and into the vein itself. The process is invasive and bears risks such as infection and bleeding. Invasive processes also require additional recovery time to repair the trauma to the skin. Venous disorders treated by endo-venous laser therapy include varicose and spider veins.

Spider veins are small, dilated blood vessels that appear red or blue under the skin. They may be in short, unconnected lines each about the size of a hair, or connected in a matted, “sunburst” pattern. While spider veins typically appear on the legs, they can also appear on the face or elsewhere.

Varicose veins are enlarged blood vessels that result from the backward flow of blood in the legs caused by damaged or diseased valves in the veins caused by a weakening in the vein's wall. The veins appear blue and bulging under the skin. The condition often leads to pain and swelling in the leg. Varicose veins are typically larger and cause more discomfort than spider veins. In the majority of cases, these leg markings can be unsightly and may be associated with symptoms such as swelling, cramping, aching, throbbing and fatigue of the legs and feet. Treatment, however, is usually sought for cosmetic reasons. In the past, when the largest superficial veins were involved, the only alternative was surgery with stripping of the defective vein, a procedure that involves making an incision in the skin and either tying off or removing the blood vessel.

The current state of the art utilizes several medical techniques for the treatment of venous disorders. These techniques include sclerotherapy, phlebectomy, electrodessication, surgical ligation and stripping, and laser surgery.

Until recently, most spider leg veins were treated with sclerotherapy. This technique involves injecting a sclerosing solution directly into the vein, causing it to collapse and form scar tissue that permanently closes it. Eventually, the vein becomes barely noticeable or invisible as it disappears into the body within a matter of weeks. A chemical solution is injected into veins to cause them to collapse and form scar tissue that permanently closes them. Nearby veins take up re-routed blood flow. Sclerotherapy requires multiple treatments to close off all affected veins. Additional treatments may be needed from time to time as new enlarged veins appear. Side effects of sclerotherapy may include slight swelling, bruising, and redness and itching at injection sites.

While sclerotherapy is successful in the majority of patients, side effects include fear of needles, skin ulceration, matting (the formation of very fine blood vessels that appear as pink patches), brown staining of the skin and, very rarely, blood clots or allergies to the solution.

Phlebectomy, also called ambulatory phlebectomy is a procedure where an enlarged vein is removed through tiny incisions along its course, in a procedure performed in an outpatient setting. The procedure can be used for large varicose veins and for spider veins. During, electrodessication an electrical current is used to seal off enlarged veins.

Surgical ligation and stripping is a procedure usually reserved for larger veins. The vein is tied off and removed by means of an incision. This procedure requires a hospital visit under sedation or general anesthesia, and commonly includes side effects such as scars, loss of skin sensation in the legs, and prolonged recovery time.

A new procedure to treat varicose veins called the Radio Frequency Closure technique, commonly referred to as the Closure technique, involves inserting a small tube called a catheter into the defective vein through a small puncture. A catheter delivers radio frequency energy to the vein wall, causing the vein to shrink and seal shut. Once the diseased vein is closed, neighboring healthy veins take over to restore normal outflow of venous blood from the legs. As normal blood flow returns, symptoms are typically reduced.

The Closure technique has distinct advantages over several other techniques because patients do not feel much if any pain either during the procedure or post-operative, and they can return to their daily activities immediately. A single treatment lasting 45 to 60 minutes can eliminate the most common leakage point of varicose veins using tumescent anesthesia. The procedure is virtually pain free.

Laser surgery is the current state of the art in the treatment of spider and varicose veins. Pulses from a laser selectively destroy target areas on enlarged veins, closing them off. Since the mid-1980's, lasers have been safely and successfully used to treat facial veins and birthmarks. Leg veins have been difficult to treat with lasers because the blood vessel walls are thicker and the blood vessels are deeper. Advances in laser technology have allowed physicians to reliably use lasers to treat leg veins, with results and side effects comparable to, or better than conventional techniques.

Endo-vascular laser procedure represents the most advanced treatment available before the discovery of the present invention. Unlike traditional laser procedures where the laser beam directly affects the skin through the laser itself with long or short pulses of light, the endo-vascular laser procedure uses a diode laser wire or fiber that is inserted directly into the vein. The laser fiber physically penetrates the skin to deliver the laser energy directly into the vein. The energy transmitted from the laser heats the varicose vein, causing it to be destroyed. The entire procedure takes approximately 30 to 60 minutes and side effects are minimal with the exception of some post-operative bruising. While patients can return to work the next day, a support stocking must be worn for 10 to 14 days following the procedure.

One of the most notable advancements in laser technology has been in the use of longer laser exposure times (or pulse durations) that decrease skin bruising, improve healing times, and enable more effective removal of larger diameter leg veins. The veins are slowly heated and coagulated, causing them to close up without the explosive rupture that can occur with shorter pulse durations. This treatment, when used with specialized methods of dermal cooling before or after the laser pulse is delivered to reduce the risk of burning, has greatly decreased the discomfort and crusting previously associated with laser procedures.

Typically, a patient will require several laser treatment sessions to effectively remove spider leg veins. A treatment session is usually 10-15 minutes long, and is performed at one-to-two month intervals to allow the damaged blood vessels to be cleared away by the body's immune system.

New laser procedures are a welcome alternative to surgery for patients with difficult to treat varicose veins, particularly for those whose condition involves the main vein trunk in the legs (the greater saphenous vein). The Endo-vascular laser procedure uses a bare laser fiber that is inserted directly into the damaged vein like a catheter through a small ¼-inch incision in the thigh. The saphenous vein is destroyed by using laser energy to heat and seal the vein from within.

Cooling devices are another important improvement in lasers used to treat venous disorders that aids in protecting the skin and makes the procedure less painful. After the laser pulse is administered to the vein, the cooling device is sprayed to reduce heat injury as the vein cools down.

Overall, most people enjoy a long period of remission after successful leg vein treatment, and can maintain the appearance of their legs with only occasional maintenance treatments. Yet while dermatologists can treat existing leg veins, they cannot prevent the body from forming new ones. Individuals with a tendency to develop leg veins should avoid standing for long periods, wear support hose for varicose veins and exercise regularly to tone the calf muscles, which helps propel the blood back to the heart and avoid pooling in the lower legs.

### SUMMARY OF THE INVENTION

The present invention is a non-invasive technique for treating varicose veins and spider veins using a dual laser. No incision is required and no catheter is used to destroy the damaged veins endo-vascularly.

The dual laser is dosed and timed so as to change the vein from a partially functioning vessel to an obsolete vessel that will be broken down and reabsorbed into the body. Dosage and timing are related to the particular size of the vessel but both fall within clearly identifiable ranges that offers therapeutic value.

The treatment parameters for venous disorders, specifically varicose veins requires the use of a pulse dye laser with fluences ranging from 7.5 to 10.0 joules per cm² coupled with 10-40 millisecond pulse width and short, medium and long delays between laser firings. This is coupled with a YAG laser having fluences between 55 and 75 joules per cm² with pulse widths varying from 15-30 milliseconds. Lasering is used in conjunction with epidermal cooling to avoid damage to the epidermis while still effectively treating the vein. This treatment regimen has been conclusively shown to not only disable and obsolete the vein but also to have beneficially affected coloration by targeting and destroying hemoglobin.

The Pulsed Dye Laser uses a concentrated beam of light that targets blood vessels in the skin. The process is referred to as selective photothermolysis. The light is converted into heat, destroying the blood vessel while leaving the surrounding skin undamaged. The laser uses yellow light, which is very safe and does not result in any long-term skin damage. The pulsed dye laser uses an organic dye as a lasing medium, usually as a liquid solution. Compared to gases and most solid state lasing media, a dye can usually be used for a much wider range of wavelengths. The wide bandwidth makes them particularly suitable for tunable lasers and pulsed lasers.

Various embodiments anticipate this treatment utilizing various YAG lasers including, but not limited to Nd and Er. An example of a dual laser that utilizes both pulsed dye and YAG lasers is the Multiplex™ by Cynosure, Inc.

Er:YAG is an acronym for Erbium-doped Yttrium Aluminium Garnet (Er:Y₃Al₅O₁₂), a compound that is used as the lasing medium for certain solid-state lasers. Er:YAG lasers typically emit light with a wavelength of 2940 nm, in the infrared. Unlike in Nd:YAG lasers, the frequency of Er:YAG lasers is at the resonant frequency of water, which leads it to being quickly absorbed; this limits its use in surgery and many other laser applications which have water present. Because of this limitation Er:YAG lasers are far less common than relatives such as Nd:YAG and Er:glass. The heat produced by the laser is proportional to the wavelength of the light.

The 2940-nm wavelength emitted by the Er:YAG laser is absorbed 12-18 times more efficiently by superficial (water-containing) cutaneous tissue than is the 10,600 nm wavelength of the traditional carbon dioxide laser. With a pulse duration of 250 μsec, a typical short-pulse Er:YAG laser ablates 5-20 μm of tissue per laser pass at a fluence of 5 J/cm² with minimal residual thermal damage (compared to 20-60 μm of tissue ablation and up to 150 μm of residual thermal damage per pass with the carbon dioxide laser). The precise tissue ablation and small zone of residual thermal damage results in faster re-epithelialization and an improved side effect profile.

Initial enthusiasm for the short-pulsed Er:YAG laser was soon tempered by poor intra-operative hemostasis and less impressive clinical improvement (reduced tissue tightening) when compared to traditional high-energy pulsed or scanned carbon dioxide laser resurfacing.

In an attempt to overcome the limitations of the short-pulsed Er:YAG laser, modulated (short-and-long-pulsed) Er:YAG systems were introduced to facilitate deeper ablation of tissue, improve hemostasis, and increase collagen remodeling. With the addition of significant coagulative properties, modulated Er:YAG systems combined precise control of ablation with the ability to induce dermal collagen formation by means of thermal injury.

Nd:YAG is an acronym for neodymium-doped yttrium aluminium garnet (Nd:Y₃Al₅O₁₂), a compound that is used as the lasing medium for certain solid-state lasers. The YAG crystal is doped with an active medium, in this case triply ionized neodymium, which replaces another element of roughly the same size, typically yttrium. Generally the crystalline host is doped with around 1% neodymium by weight.

Nd:YAG lasers are optically pumped using a flashlamp or laser diodes. They are one of the most common types of laser, and are used for many different applications.

Nd:YAG lasers typically emit light with a wavelength of 1064 nm, in the infrared. However, there are also transitions near 940, 1120, 1320, and 1440 nm. Nd:YAG lasers operate in both pulsed and continuous mode. Pulsed Nd:YAG lasers are typically operated in the so called Q-switching mode: An optical switch is inserted in the laser cavity waiting for a maximum population inversion in the neodymium ions before it opens. Then the light wave can run through the cavity, depopulating the excited laser medium at maximum population inversion. In this Q-switched mode output powers of 20 megawatts and pulse durations of less than 10 nanoseconds are achieved.

Nd:YAG absorbs mostly in the bands between 730-760 nm and 790-820 nm. Krypton flashlamps, with high output at those bands, are therefore more efficient for pumping Nd:YAG lasers than the xenon lamps, which produce more white light and hence more energy therefore goes wasted.

The amount of the neodymium dopant in the material varies according to its use. For continual wave output, the doping is significantly lower than for pulsed lasers. The lightly doped CW rods can be optically distinguished by being less colored, almost white, while higher-doped rods are pink-purplish.

Other common host materials for neodymium are: YLF (yttrium lithium fluoride, 1047 and 1053 nm), YVO₄ (yttrium vanadate, 1064 nm), and glass. A particular host material is chosen in order to obtain a desired combination of optical, mechanical, and thermal properties. Nd:YAG lasers and variants are pumped either by flash lamps, continuous gas discharge lamps, or near-infrared laser diodes (DPSS lasers).

The method requires heating the blood with the pulsed dye laser with a sub-purpuric dose to create methomoglobin (metHb), and oxidized form of hemoglobin (Hb) formed during thermal denaturation. The metHb is subsequently targeted by the YAG laser. A light anesthetic may sometimes be beneficial for the comfort of the patient. The metHb more easily absorbs the Nd-YAG laser energy because of enhanced absorption at 1064 nm. Raised vessels dissipate heat faster and can be harder to treat. A 5 mm to 10 mm spot size is utilized, with a preferred spot size of 7 mm to 10 mm.

After an appropriate pulse of energy delivers a beneficial amount of energy to heat the blood, the pulse dye laser is shut off and, after a short delay but before the blood travels outside the range of the YAG laser, the Nd-YAG laser fires to effectuate the destruction of the target vessel. The vessel shrinks and seals itself off at both ends. The hemoglobin in the vessel is also destroyed and, because the vessel shrinks instead of bursts, no bruising occurs and the healing process is accelerated. Dermal cooling is utilized at the end of or during the treatment to minimize the risk of thermal injury. Typically, the procedure is completely effective the first treatment and no follow-up treatments are necessary. The following table provides prophetic examples of ranges that the inventor believes will provide the optimal results and are based upon previous work.

TABLE I
Prophetic Laser Setting Ranges Per Vessel Diameter

| | Pulse Dye Laser | | | Nd-YAG Laser | |
|---|---|---|---|---|---|
| Vessel | | Pulse | | Pulse | |
| diameter | Fluence | Width | Laser Delay | Fluence | Width |
| range (mm) | (J/cm2) | (ms) | (ms) | (J/cm2) | (ms) |
| 0 to 2 | 7.0 to 8.5 | 10 to 20 | 50, 150 | 60 to 75 | 15 to 30 |
| 1 to 4 | 7.5 to 9.0 | 10 to 20 | 50, 150 | 65 to 75 | 15 to 30 |
| 3 to 6 | 8.0 to 9.5 | 10 to 40 | 50, 150 | 70 to 80 | 15 to 30 |
| 5 to 10 | 9.0 to 10.0 | 20 to 40 | 50, 150, 250 | 70 to 80 | 15 to 30 |
| >7 | 9.5 to 10.0 | 20 to 40 | 50, 150, 250 | 75 to 80 | 15 to 30 |

What is claimed is:

1. A method for treating vascular disorders comprising the use of a pulse dye laser and a YAG laser to trans-dermally treat the targeted vessel.

2. The method of claim 1, wherein varicose veins are eliminated.

3. The method of claim 1, wherein spider veins are eliminated.

4. The method of claim 1, wherein the appearance of varicose veins are minimized.

5. The method of claim 1, wherein the appearance of spider veins are minimized.

6. The method of claim 1, wherein said pulsed dye laser delivers a sub-purpuric dose of energy.

7. The method of claim 6, wherein said dose of energy delivered by said pulsed dye laser is sufficient to convert Hb to metHb within the target vessel.

8. The method of claim 7, wherein said pulsed dye laser fluences range from 7 to 10 joules per square cm.

9. The method of claim 8, wherein said pulsed dye laser has a pulse width between 10 and 40 milliseconds.

10. The method of claim 1, wherein said YAG laser is an Nd-YAG laser.

11. The method of claim 11, wherein said YAG laser fluences range from 55 to 85 joules per square cm.

12. The method of claim 3, wherein said YAG laser has a pulse width is between 1 and 50 milliseconds.

13. The method of claim 1, wherein the pulse dye laser is fired before the YAG laser.

14. The method of claim 14, wherein the time delay between the firing of the pulse dye laser and the YAG laser is less than 250 milliseconds.

15. The method of claim 1, wherein said YAG laser is fired at a point in time and at a point on the vessel so as to affect the metHb created by said pulse dye laser.

16. The method of claim 1, wherein said pulse dye laser fluence and pulse width settings are determined by a method comprising correllating vessel size and depth in the skin with laser fluence and pulse width variables.

17. The method of claim 1, wherein said YAG laser fluence and pulse width settings are determined by a method comprising correllating vessel size and depth in the skin with laser fluence and pulse width variables.

18. A method for treating vascular disorders comprising the use of a pulse dye laser followed by the use of an Nd-YAG laser to affect a targeted vessel, said pulse dye laser and said Nd-YAG laser being utilized trans-dermally to said vessel and further comprising the method of using said pulse dye laser to deliver sufficient energy to the blood within said targeted vessel to convert a quantity of Hb to metHb so as to create sufficient heat within said targeted vessel when said metHb is affected by the energy delivered by said Nd-YAG laser.

19. The method of claim 18, wherein said pulsed dye laser is operated with fluences from 7 to 10 joules per square cm and at a pulse width between 10 and 40 milliseconds.

20. The method of claim 18, wherein said Nd-YAG laser is operated with fluences from 55 to 75 joules per square cm and at a pulse width is between 10 and 40 milliseconds.

21. The method of claim 18, wherein said Nd-YAG laser is fired from less than 250 milliseconds after the termination of the firing of said pulse dye laser.

22. The method of claim 21, wherein said pulse dye laser delivers a sub-purpuric dose of energy.

23. The method of claim 22, wherein said pulse dye laser and said Nd-YAG laser are operated at settings that correlate to vessel size and depth in the skin.

24. The method of claim 23, wherein the epidermis is cooled during the procedure.

25. The method of claim 23, wherein the epidermis is cooled following the procedure.

26. A method for treating vascular disorders comprising:
(a) the step of treating a target vessel with energy from a pulse dye laser to convert hemoglobin within said target vessel to;
(b) the step of treating the target vessel and Nd-YAG laser to affect the targeted vessel;
(c) The step of pulse dye laser and said Nd-YAG laser being utilized trans-dermally to treat the vessel, and wherein said pulsed dye laser utilizes fluences from 7 to 10 joules per square cm and a pulse width between 10 and 40 milliseconds and said Nd-YAG laser is operated utilizing fluences from 55 to 75 joules per square cm and a pulse width is between 10 and 40 milliseconds and said Nd-YAG laser is fired less than 250 milliseconds after the termination of the firing of the pulse dye laser, said pulse dye laser delivers a sub-purpuric dose of energy, wherein said pulse dye laser and said Nd-YAG laser are operated at settings that correlate to vessel size and depth in the skin and said time delay between firings is optimized to permit the targeting of the pulse dye laser created metHb by said Nd-YAG laser.
